SEQUENCE LISTING (2 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

(19) United States
(12) Patent Application Publication
(10) Pub. No.: US 2022/0087233 A1
(43) Pub. Date: Mar. 24, 2022

(54) NON-HUMAN ANIMAL MODELS OF SÉZARY SYNDROME

(71) Applicants: (INSERM) Institut National de la Santé et de la Recherche Médicale), Paris (FR); Université de Paris, Paris (FR); Assistance Publique-Hôpitaux de Paris (APHP), Paris (FR)

(72) Inventors: Anne MARIE-CARDINE, Paris (FR); Jean-Luc POYET, Paris (FR); Justine HABAULT, Paris (FR); Armand BENSUSSAN, Paris (FR); Martine BAGOT, Paris (FR)

(21) Appl. No.: 17/424,973

(22) PCT Filed: Jan. 24, 2020
(86) PCT No.: PCT/EP2020/051750

§ 371 Date: Jul. 22, 2021

(30) Foreign Application Priority Data
Jan. 25, 2019 (EP) 19305098.6

(51) Int. Cl. A01K 67/027; A61K 49/00

(52) U.S. Cl. CPC A01K 67/0271; A61K 49/0008; A01K 2227/105; A01K 2267/0331; A01K 2207/12

(57) ABSTRACT

Sézary syndrome is a rare, aggressive, and leukemic form of cutaneous T-cell lymphoma (CTCL) characterized by erythroderma associated with generalized peripheral lymphadenopathy and circulating clonal malignant T cells called Sézary cells. Current animal models of Sézary syndrome are not satisfactory since no cutaneous symptoms or occurrence of metastases could be observed. Now the inventors developed a new non-human animal model that repeat the major cutaneous symptoms of the human disease. This model could be suitable for screening new drugs and biomarkers of the disease.

### FIELD OF THE INVENTION

The present invention relates to non-human animal models of Sézary syndrome and uses thereof.

### BACKGROUND OF THE INVENTION

Sézary syndrome is a rare, aggressive, and leukemic form of cutaneous T-cell lymphoma (CTCL) characterized by erythroderma associated with generalized peripheral lymphadenopathy and circulating clonal malignant T cells called Sézary cells. Basically, in the current International Society for Cutaneous Lymphomas (ISCL)/European Organisation for Research and Treatment of Cancer (EORTC) TNMB staging classification, the diagnosis of Sézary syndrome requires erythroderma with a positive T-cell clone in the peripheral blood associated with at least one B2 criterion including the identification of more than 1,000 Sézary cells/mm3 in the blood as determined by cytomorphologic analysis. Patients with Sézary syndrome have a bad prognosis, with a 5-year overall survival varying from 24% to 43%. There is no curative treatment and available systemic treatments have often short-lived responses, with relapses after few weeks or months. The treatment options are based on the stage of the disease. Given the leukemic involvement in Sézary syndrome, the treatment is generally systemic. It can be given alone or in a combination of skin-based therapy. Stage IVA (no visceral involvement) patients are usually treated with extracorporeal phototherapy (ECP) combined with biological response modifiers (retinoids and interferons). Other alternatives include low-dose methotrexate and histone deacetylase inhibitors (vorinostat and romidepsin). Various combinations of the above can be used along with skin-directed therapy.

The utility of primary Sézary Syndrome xenografts as a platform to study cancer biology and to develop novel therapeutic and diagnostic approaches to cancer has been demonstrated. Studies have shown that these tumors maintain the main features of the originating cancer; hence, it is believed that their use in preclinical studies reproduces more accurately the clinical scenario compared with studies done with cell lines. However, currently, these types of models have not been successfully generated for Sézary Syndrome and the rare attempts were partially satisfactory since no cutaneous symptoms and occurrence of metastases were observed.

### SUMMARY OF THE INVENTION

As defined by the claims, the present invention relates to non-human animal models of Sézary syndrome and uses thereof.

### DETAILED DESCRIPTION OF THE INVENTION

The first object of the present invention relates to a method of producing an animal model of Sézary syndrome comprising the steps of i) engrafting an amount of peripheral blood mononuclear cells (PBMC) obtained from a patient suffering from the disease in an immunodeficient non-human animal and ii) promoting the expansion and maintaining the survival of tumor cells by weekly administering to the animal an amount of IL-2 and IL-7.

As used herein, the term “Sézary syndrome” has its general meaning in the art and refers to a rare, aggressive, and leukemic form of cutaneous T-cell lymphoma (CTCL) characterized by erythroderma associated with generalized peripheral lymphadenopathy and circulating clonal malignant T cells called Sézary cells.

As used herein, the term “subject” denotes a mammal or an animal such as a rodent, a feline, a canine, and a primate. Particularly, the subject according to the invention is a rodent.

As used herein, the term “peripheral blood mononuclear cell” or “PBMC” has its general meaning in the art and refers to a population of white blood cells having a round nucleus, which has not been enriched for a given sub-population. Typically, these cells can be extracted from whole blood using Ficoll, a hydrophilic polysaccharide that separates layers of blood, with the PBMC forming a cell ring under a layer of plasma. Such procedures are known to the expert in the art. A typical protocol for isolating PBMC from a blood sample obtained from a patient suffering from Sézary syndrome is described in the EXAMPLE. Only patients who presented a % of tumor cells ≥95% among their CD4+ T cell population are eligible for preparing the PBMC that are engrafted in the immunodeficient animal.

As used herein, the term “immunodeficient non-human animal” refers to a non-human animal (e.g., mouse) characterized by one or more of: a lack of functional immune cells, such as T cells and B cells; a DNA repair defect; a defect in the rearrangement of genes encoding antigen-specific receptors on lymphocytes; and a lack of immune functional molecules such as IgM, IgG1, IgG2a, IgG2b, IgG3 and IgA. In some embodiments, the immunodeficient non-human animal is an immunodeficient mouse. More particularly, the immunodeficient mouse is a NOD SCID gamma (NSG) mouse as described in detail in Shultz et al., J. Immunol., 174:6477-6489, 2005. The term “severe combined immune deficiency (SCID)” refers to a condition characterized by absence of T cells and lack of B cell function. The terms “NOD scid gamma” and “NSG” are used interchangeably herein to refer to a well-known immunodeficient mouse strain NOD.Cg-Prkdcscid NSG mice combine multiple immune deficits from the NOD/ShiLtJ background, the severe combined immune deficiency (scid) mutation, and a complete knockout of the interleukin-2 receptor gamma chain. As a result, NSG mice lack mature T, B and NK cells, and are deficient in cytokine signaling. NSG mice are characterized by lack of IL2R-γ (gamma c) expression, no detectable serum immunoglobulin, no haemolytic complement, no mature T lymphocytes, and no mature natural killer cells.

The inventors have found that a an amount of about 1×10⁶ PBMC cells, 2×10⁶ PBMC cells, 3×10⁶ PBMC cells, 4×10⁶ PBMC cells, 5×10⁶ PBMC cells, 6×10⁶ PBMC cells, 7×10⁶ PBMC cells, 8×10⁶ PBMC cells, 9×10⁶ PBMC cells, 10×10⁶ PBMC cells, 11×10⁶ PBMC cells, 12×10⁶ PBMC cells, 13×10⁶ PBMC cells, 14×10⁶ PBMC cells, 15×10⁶ PBMC cells, 16×10⁶ PBMC cells, 17×10⁶ PBMC cells, 18×10⁶ PBMC cells, 19×10⁶ PBMC cells, 20×10⁶ PBMC cells, 21×10⁶ PBMC cells, 22×10⁶ PBMC cells, 23×10⁶ PBMC cells, 24×10⁶ PBMC cells, 25×10⁶ PBMC cells, 26×10⁶ PBMC cells, 27×10⁶ PBMC cells, 28×10⁶ PBMC cells, 29×10⁶ PBMC cells, or 30×10⁶ PBMC cells may be used for engrafting the cells in the immunodeficient animal. Preferably, an amount of about 20×10⁶ of PBMC is engrafted in the immunodeficient animal.

As used herein, the term “about,” as applied to one or more values of interest, refers to a value that is similar to a stated reference value. In some embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction of the stated reference value unless otherwise stated or otherwise evident from the context.

Typically, the engraftment is performed by the caudal intravenous injection of the PBMC as described in the EXAMPLE.

As used herein, the term “IL-2” has its general meaning in the art and refers to the interleukin-2 that is typically required for T-cell proliferation and other activities crucial to regulation of the immune response. An exemplary human amino acid sequence for IL-2 is represented by SEQ ID NO:1.

>sp|P60568|IL2_HUMAN Interleukin-2
OS = Homo sapiens OX = 9606 GN = IL2 PE = 1 SV = 1
SEQ ID NO: 1
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

As used herein, the term “IL-7” has its general meaning in the art and refers to the interleukin-2 that is in particular described as a hematopoietic growth factor capable of stimulating the proliferation of lymphoid progenitors. An exemplary human amino acid sequence for IL-2 is represented by SEQ ID NO:2.

>sp|P13232|IL7_HUMAN Inter1eukin-7
OS = Homo sapiens OX = 9606 GN = IL7 PE = 1 SV = 1
SEQ ID NO: 2
MFHVSFRYIFGLPPLILVLLPVASSDCDIEGKDGKQYESVLMVSIDQLLD
SMKEIGSNCLNNEFNFFKRHICDANKEGMFLFRAARKLRQFLKMNSTGDF
DLHLLKVSEGTTILLNCTGQVKGRKPAALGEAQPTKSLEENKSLKEQKKL
NDLCFLKRLLQEIKTCWNKILMGTKEH

The inventors have found that a an amount of IL-2 of about 80 UI/ml, 81 UI/ml, 82 UI/ml, 83 UI/ml, 84 UI/ml, 85 UI/ml, 86 UI/ml, 87 UI/ml, 90 UI/ml, 91 UI/ml, 92 UI/ml, 93 UI/ml, 94 UI/ml, 95 UI/ml, 96 UI/ml, 97 UI/ml, 98 UI/ml, 99 UI/ml, 100 UI/ml, 101 UI/ml, 102 UI/ml, 103 UI/ml, 1040 UI/ml, 105 UI/ml, 106 UI/ml, 107 UI/ml, 108 UI/ml, 109 UI/ml, 110 UI/ml, 111 UI/ml, 112 UI/ml, 113 UI/ml, 114 UI/ml, 115 UI/ml, 116 UI/ml, 117 UI/ml, 118 UI/ml, 119 UI/ml, or 120 UI/ml may be used. Preferably an amount of 100 UI/ml is used.

The inventors have found that a an amount of IL-7 of about 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, or 20 ng/ml may be used. Preferably an amount of 15 ng/ml is used.

In some embodiments, the IL-2 and IL-7 are administered to the non-human anima as a mix.

In some embodiments, the IL-2 and IL-7 are weekly administered for 3, 4, 5, 6, 7, 8, 9, 10, 11, 12, 13 or 15 weeks depending on the condition of the animal that may require sacrifice for ethic reasons.

A further object of the present invention relates to a method of studying Sézary Syndrome, comprising providing the animal model of the invention, and evaluating at least one parameter of engrafted human tumor cells.

In some embodiments, the parameter comprises the presence or absence of a biomarker. In some embodiments, the biomarker comprises one or more tumor markers (e.g. KIR3DL2). In some embodiments, the biomarker is a gene expression signature. Typically, the presence or absence of the biomarker is evaluated in a sample obtained from the animal model. Typically, the sample may be a blood sample or a skin sample (e.g. biopsy). Typically, any immunoassay well known in the art may be suitable for the in vitro evaluation and typically involves ELISA, immunochemistry (IHC) or flow cytometry. Typically flow cytometry as described in the EXAMPLE is performed.

Thus, the method of the present invention is particularly suitable for identifying a biomarker for Sézary syndrome.

A further object of the present invention relates to a method of evaluating the survival of the engrafted tumor cells, comprising providing an animal model of the invention, and evaluating said survival.

Evaluation of tumor cell survival following implantation can be carried out ex vivo or in vivo. For example, evaluation of tumor cell survival may be carried out in vivo with an imaging modality selected from among ultrasound imaging, fluorescence molecular tomography (FMT), and magnetic resonance imaging (e.g., anatomical MM, diffusion MM, Mill spectroscopy, dynamic contrast enhanced (DCE) MRI). For example, evaluation of tumor cell survival may be carried out ex vivo in a sample obtained from the animal model. Typically, the sample may be a blood sample or a skin sample (e.g. biopsy). Typically, any immunoassay well known in the art may be suitable for the in vitro evaluation and typically involves ELISA, immunochemistry (IHC) or flow cytometry. Typically flow cytometry as described in the EXAMPLE is performed.

In some embodiments, a test substance is administered to the animal before, during, and/or after engraftment of the tumor cells and the response of the tumor cells to the test substance is evaluated ex vivo or in vivo.

In some embodiments, a cancer treatment is administered to the animal before, during, and/or after engraftment of the tumor cells and the response of the tumor cells to the treatment is evaluated ex vivo or in vivo. For example, tumor cell survival may be evaluated ex vivo or in vivo in response to a cancer treatment or to a test substance. Optionally, a combination of test substances is administered and its effect is evaluated. In some embodiments, the test substance is a chemotherapeutic agent or other anti-cancer agent. However, the test substance may be a non-anti-cancer agent. Typically, the test substance of the invention may be selected from a library of substances previously synthesised, or a library of substances for which the structure is determined in a database, or from a library of substances that have been synthesised de novo. The test substance may be selected from the group of (a) proteins (including antibodies) or peptides, (b) nucleic acids and (c) organic or chemical substances.

Thus, the method as above described is particularly suitable for screening a drug useful for the treatment of Sézary syndrome.

Accordingly, a further object of the present invention relates to a method for screening a drug suitable for the treatment of Sézary syndrome comprising the steps of i) administering the animal model as herein disclosed with an amount of a test substance, and ii) selecting the test substance that is able to kill or to reduce the amount of the tumor cells in said animal.

In some embodiments, the survival of tumor cells in the animal administered with the test substance is compared with the survival of the tumor cells in an animal that was not administered with the test substance, wherein a higher survival observed in the animal administered with the test substance that the survival observed with the animal that was not administered with the test substance indicates that the test substance is useful for killing or reducing the amount of tumor cells.

A further object of the present invention relates to a method for screening potential treatments for Sézary syndrome in a subject, comprising producing the animal model as herein disclosed; administering a candidate treatment to the animal before, during, or after said engraftment; and evaluating at least one parameter of the tumor cells that is associated with cancer treatment efficacy or lack of efficacy.

The candidate treatment may be, for example, a chemotherapeutic treatment or other anti-cancer treatment, a radiation treatment, or any combination of two or more anti-cancer treatments. The parameter(s) evaluated may be parameters of the tumor cells and/or the animal that provide information as to whether the candidate treatment is effective in treating the cancer. For example, the at least one parameter may comprise tumor cell survival rate or tumor burden. In some embodiments, the evaluation comprises imaging at least a portion of the animal to determine the response of the one or more human tumor cells to the candidate treatment. Imaging can be carried out, for example, with an imaging modality selected from among one or more of, ultrasound imaging, fluorescence molecular tomography (FMT), and magnetic resonance imaging (e.g., anatomical MM, diffusion MM, MRI spectroscopy, dynamic contrast enhanced (DCE) MRI). In some embodiments, skin biopsies as described in the EXAMPLE may be performed.

In some embodiments a plurality of animal models is produced and a different candidate treatment is administered to each animal. In some embodiments, in order to obtain information concerning effective dose or optimum dose, a different dose of the same candidate treatment can be administered to each animal.

In some embodiments of the screening method, the method further comprises selecting and administering the candidate treatment to the subject if the results of the evaluation are consistent with treatment efficacy.

A further object of the present invention thus related to a method for treating Sézary syndrome in a subject, comprising selecting a candidate treatment from among a plurality of candidate treatments, and administering the selected treatment to the subject, wherein the selected candidate treatment has been determined to be effective in treating Sézary syndrome in the non-human animal model herein disclosed.

The invention will be further illustrated by the following figures and examples. However, these examples and figures should not be interpreted in any way as limiting the scope of the present invention.

### FIGURES

FIG. 1: H&E coloration of skin biopsies from human and mouse. Cryosections were prepared from skin fragments of a healthy donor (A), Sézary patient (B), untreated NSG mouse (C) or NSG mouse engrafted with Sézary patient PBMC (D) and subjected to H&E coloration.

FIG. 2: Flow cytometry analysis of the T cell tumor burden pre- and post-injection. Immunostaining was performed on Sézary patient blood (A) or on cells extracted from the skin of the corresponding recipient NSG mouse (B). Tumor CD4⁺ T cells are identified through expression of KIR3DL2 and TCR-Vβ clonality.

### EXAMPLE

Material and Methods:

Cells

PBMC were isolated from Sézary syndrome (SS) patients heparinized venous blood by gradient centrifugation on lymphocytes separation medium (LSM; EuroBio). Cells were washed once in phosphate buffer saline (PBS; Invitrogen) and resuspended at a concentration of 20×10⁶ cells/200 μl of saline solution (0.9% NaCl).

Generation of a Sézary Mouse Model

Sézary patient tumor burden was first evaluated by flow cytometry. Only patients who presented a % of tumor cells ≥95% among their CD4⁺ T cell population were selected for the next experimental steps. PBMC were then prepared and immediately processed for engraftment. Four to twelve-week old NOD/SCID/gamma (NSG) female mice were engrafted by caudal intravenous injection of 20×10⁶ Sézary patient PBMC. IL-2 and IL-7 (cytokine mix; Peprotech) were added to the cells at concentrations of 100 UI/ml and 15 ng/ml, respectively. Once per week, a fresh cytokine mix was re-injected.

Mice well-being was monitored each other day in terms of weight, behaviour and skin appearance (after hair removal with a depilatory cream). Mice were sacrificed when abnormal behaviour, skin damages and/or inherent itching became incompatible with the animal well-being.

Lymphocytes Extraction from Skin Biopsies

Biopsies were dilacerated and skin fragments were incubated in RPMI 1640 culture medium supplemented with 2 mg/ml of collagenase II (Sigma-Aldrich) at 37° C. for 30 min. After washes, skin debris were eliminated by passing the mixture through a 100 μm nylon cell strainer and the collected cells were analyzed by flow cytometry as described below.

Flow Cytometry

Blood or extracted cutaneous cells were immunolabeled with the following mix of fluorochrome-conjugated antibodies to allow detection of the malignant CD4⁺ T cells: TCRVβ-FITC/KIR3DL2-PE/CD3-PC5/CD4-PC7/CD45-Pacific Blue. Tumor cells were identified as CD3⁺ TCRVβ⁺ CD45⁺ CD4⁺ KIR3DL2⁺ cells. Cells were acquired on a cytometer (CytoFlex; Beckman Coulter) and data analyzed using FlowJo software.

Haematoxylin & Eosin (H&E) Coloration

Immediately after isolation, skin biopsies were immersed in 4% paraformaldehyde, included in paraffin and stored at 4° C. until use. Five μm cryosections were cut using a cryostat, air-dried, fixed in acetone, washed and incubated sequentially in haematoxylin solution, 0.08% NH₄OH and 0.2% eosin solution. Washes were performed between each step of the procedure. After a final wash in ethanol, pictures were acquired on a Leica DMRB microscope.

Results

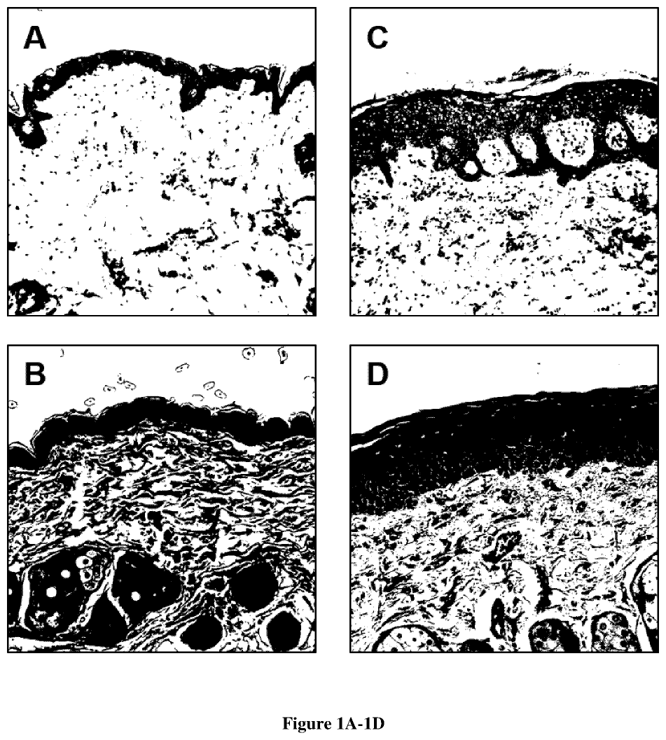

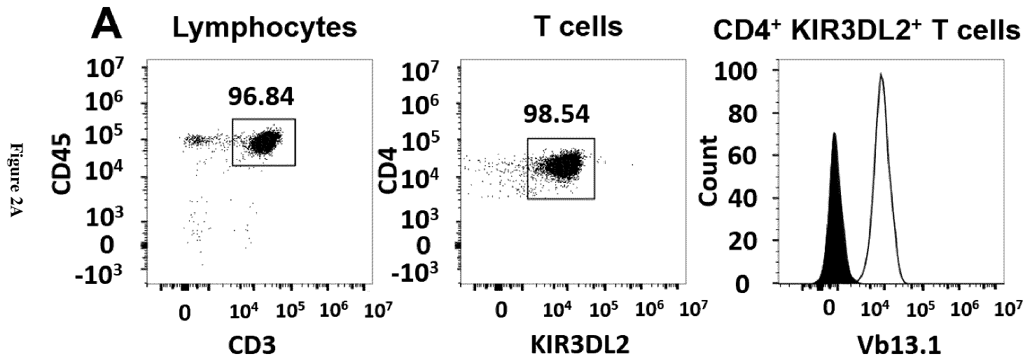

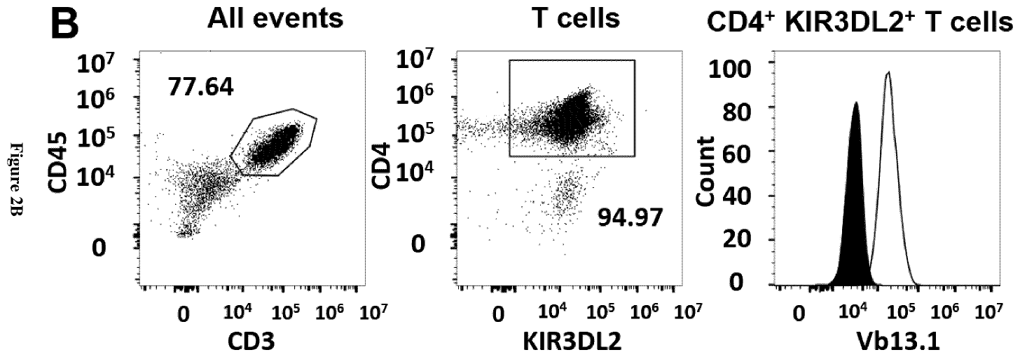

Sézary syndrome is an advanced and aggressive form of cutaneous T cell lymphoma characterized by the presence of tumor T cells in the blood and skin. In patients' skin, the presence of malignant T cell infiltrates results in keratinocytes hyper-proliferation leading to epidermis thickening (FIGS. 1A and 1B). A similar cutaneous pattern was observed in NSG mice following injection of Sézary patient PBMC when compared to non-treated mice (FIGS. 1C and 1D). In addition, cells extraction performed on skin biopsies from engrafted mice led to the detection of malignant CD4⁺ T cells exhibiting TCR-Vβ rearrangement and KIR3DL2-positivity identical to the one detected on the tumor CD4⁺ T cell clone present within the originally inoculated PBMC (FIGS. 2A and 2B). This clearly indicates that the majority of the human T lymphocytes encountered in the skin of the engrafted mice corresponded to the patient malignant T cell clone. Finally, in some mice, the presence of circulating tumor cells was also observed in the blood stream at the time of sacrifice (data not shown). In our 5 sets of experiments performed on a total of 31 mice, only one mouse showed a GVHD reaction while all other animals developed cutaneous manifestations (development of patches, plaques and/or erythrodermia). However, no metastases were observed in the main organs (lung, liver or heart), while a mild splenomegaly was evidenced in some subjects (n=10/31) at sacrifice.

### REFERENCES

Throughout this application, various references describe the state of the art to which this invention pertains. The disclosures of these references are hereby incorporated by reference into the present disclosure.

1. A process of producing an animal model of Sézary syndrome comprising the steps of i) engrafting an amount of peripheral blood mononuclear cells (PBMC) obtained from a patient suffering from the disease in an immunodeficient non-human animal and ii) promoting the expansion and maintaining the survival of tumor cells by weekly administering to the animal an amount of IL-2 and IL-7.

2. The process of claim 1 wherein the PBMC are obtained from a patient that presents a % of tumor cells ≥95% among their CD4+ T cell population.

3. The process of claim 1 wherein the immunodeficient non-human animal is is a NOD SCID gamma (NSG) mouse.

4. The process of claim 1 wherein an amount of about 1×10⁶ PBMC cells, 2×10⁶ PBMC cells, 3×10⁶ PBMC cells, 4×10⁶ PBMC cells, 5×10⁶ PBMC cells, 6×10⁶ PBMC cells, 7×10⁶ PBMC cells, 8×10⁶ PBMC cells, 9×10⁶ PBMC cells, 10×10⁶ PBMC cells, 11×10⁶ PBMC cells, 12×10⁶ PBMC cells, 13×10⁶ PBMC cells, 14×10⁶ PBMC cells, 15×10⁶ PBMC cells, 16×10⁶ PBMC cells, 17×10⁶ PBMC cells, 18×10⁶ PBMC cells, 19×10⁶ PBMC cells, 20×10⁶ PBMC cells, 21×10⁶ PBMC cells, 22×10⁶ PBMC cells, 23×10⁶ PBMC cells, 24×10⁶ PBMC cells, 25×10⁶ PBMC cells, 26×10⁶ PBMC cells, 27×10⁶ PBMC cells, 28×10⁶ PBMC cells, 29×10⁶ PBMC cells, or 30×10⁶ PBMC cells is used.

5. The process of claim 1 wherein an amount of about 20×10⁶ of PBMC is engrafted in the immunodeficient animal.

6. The process of claim 1 wherein an amount of IL-2 of about 80 UI/ml, 81 UI/ml, 82 UI/ml, 83 UI/ml, 84 UI/ml, 85 UI/ml, 86 UI/ml, 87 UI/ml, 90 UI/ml, 91 UI/ml, 92 UI/ml, 93 UI/ml, 94 UI/ml, 95 UI/ml, 96 UI/ml, 97 UI/ml, 98 UI/ml, 99 UI/ml, 100 UI/ml, 101 UI/ml, 102 UI/ml, 103 UI/ml, 1040 UI/ml, 105 UI/ml, 106 UI/ml, 107 UI/ml, 108 UI/ml, 109 UI/ml, 110 UI/ml, 111 UI/ml, 112 UI/ml, 113 UI/ml, 114 UI/ml, 115 UI/ml, 116 UI/ml, 117 UI/ml, 118 UI/ml, 119 UI/ml, or 120 UI/ml is used.

7. The process of claim 1 wherein an amount of IL-2 of about 100 UI/ml is used.

8. The process of claim 1 wherein an amount of IL-7 of about 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, or 20 ng/ml is used.

9. The process of claim 1 wherein an amount of IL-7 of about 15 ng/ml is used.

10. A non-human animal model of Sézary syndrome obtainable by a process of producing an animal model, said process comprising the steps of i) engrafting an amount of peripheral blood mononuclear cells (PBMC) obtained from a patient suffering from the disease in an immunodeficient non-human animal and ii) promoting the expansion and maintaining the survival of tumor cells by weekly administering to the animal an amount of IL-2 and IL-7.

11. A process for screening drugs or biomarkers comprising the step of administering to the animal model of claim 10 an amount of a test substance.

12. A process for screening a drug suitable for the treatment of Sézary syndrome comprising the steps of i) administering the animal model of claim 10 with an amount of a test substance, and ii) selecting the test substance that is able to kill or to reduce the amount of the tumor cells in said animal.

13. The process of claim 12, wherein the survival of tumor cells in the animal administered with the test substance is compared with the survival of tumor cells in an animal that was not administered with the test substance, wherein a higher survival observed in the animal administered with the test substance than the survival observed with the animal that was not administered with the test substance indicates that the test substance is useful for killing or reducing the amount of tumor cells.

14. A process for screening potential treatments for Sézary syndrome in a subject, comprising producing the animal model of claim 10; administering a candidate treatment to the animal before, during, or after the step of engrafting; and evaluating at least one parameter of the tumor cells that is associated with cancer treatment efficacy or lack of efficacy.

15. A process for treating Sézary syndrome in a subject, comprising selecting a candidate treatment from among a plurality of candidate treatments, and administering the selected treatment to the subject, wherein the selected candidate treatment has been determined to be effective in treating Sézary syndrome in the non-human animal model of claim 10.
